(19) United States
(12) Patent Application Publication
(10) Pub. No.: US 2023/0338320 A1
(43) Pub. Date: Oct. 26, 2023

(54) ORAL FILM COMPOSITION COMPRISING LEVOTHYROXINE

(71) Applicant: Wockhardt Limited, Aurangabad (IN)

(72) Inventors: Jack Aurora, Kentwood, MI (US); Moinuddin Rashid Syed, Aurangabad (IN); Rajendra Gawali, Ahmednagar (IN)

(73) Assignee: Wockhardt Limited, Aurangabad (IN)

(21) Appl. No.: 17/790,522

(22) PCT Filed: Nov. 23, 2020
(86) PCT No.: PCT/IN2020/050975

§ 371 Date: Jul. 1, 2022

(30) Foreign Application Priority Data
Nov. 22, 2019 (IN) 201921047820

(51) Int. Cl. A61K 31/198; A61K 9/00; A61K 47/32; A61K 47/34; A61K 47/36; A61K 9/70; A61K 47/10; A61K 47/38; A61K 47/12

(52) U.S. Cl. CPC A61K 31/198; A61K 9/006; A61K 9/7007; A61K 47/10; A61K 47/12; A61K 47/32; A61K 47/34; A61K 47/36; A61K 47/38

(57) ABSTRACT

The invention relates to a pharmaceutical composition in the form of an oral film comprising levothyroxine and a film forming polymer. The oral film can be of various types such as and including a mouth dissolving or soluble film, non-mucoadhesive film, fast disintegrating film, buccal film, mucoadhesive film, sublingual film, edible film or wafer. The invention also relates to a process of preparation of such oral film compositions.

### PRIORITY DETAILS

This application claims priority from the Indian provisional application No. IN 201921047820 filed on Nov. 22, 2020.

### TECHNICAL FIELD OF THE INVENTION

The invention relates to a pharmaceutical composition in the form of an oral film comprising levothyroxine and a film forming polymer. The oral film can be of various types such as and including a mouth dissolving or soluble film, non-mucoadhesive film, fast disintegrating film, buccal film, mucoadhesive film, sublingual film, edible film or wafer. The invention also relates to a process of preparation of such oral film compositions.

### BACKGROUND OF THE INVENTION

Oral administration of active substances in the form of tablets or capsules is extensively applied in the pharmaceutical and nutritional supplements industry. Conventional oral dosage forms such as tablets and capsules are meant to be swallowed whole or chewed with sufficient amounts of liquid to deliver the medication into the gastro-intestinal tract. Liquids syrups and suspensions are an alternative to solid dosage forms, however in these dosage forms dosing accuracy cannot be ensured. Tablets may be formulated so as to be quick dissolving (orally disintegrating tablets) such that when placed on tongue they disintegrate rapidly in the oral cavity. Generally quick dissolving tablets are formed using complex multi-step manufacturing processes, thus making it cumbersome to manufacture.

Levothyroxine, also known as L-thyroxine, synthetic T4, or 3,5,3′,5′-tetraiodo-L-thyronine, is a synthetic form of thyroxine, which is used as a hormone substitute for patients with thyroid conditions such as hypothyroidism as well as conditions in which the thyroid gland becomes enlarged, causing swelling of the neck.

Levothyroxine Sodium is the sodium salt of levothyroxine, a synthetic levoisomer of thyroxine (T4) that is similar to the endogenous hormone produced by the thyroid gland. Levothyroxine Sodium has IUPAC Name as sodium;(2S)-2-amino-3-[4-(4-hydroxy-3,5-diiodophenoxy)-3,5-diiodophenyl]propanoic acid. Levothyroxine (T4) sodium has an empirical formula of C₁₅H₁₀I₄N NaO₄• H₂O, molecular weight of 798.86 (anhydrous), and structural formula as shown below:

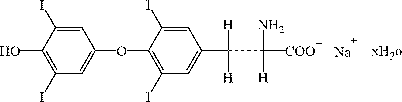

Levothyroxine is approved in various dosage forms by USFDA as oral capsule, oral tablet, oral solution, IV Powder and IV solution.

Oral films are thin films containing a pharmaceutically active substance which are placed directly in the oral cavity or applied to the oral mucosa or placed directly on to the top or under the tongue, where such films dissolve and the contained drug gets absorbed from there. These are, in particular, thin active substance-containing films based on polymers which, when applied to a mucous membrane, in particular the oral mucosa, release the active ingredient directly into the latter. These oral thin films are usually not sticky to the outside. The active ingredient may be dissolved, emulsified or dispersed in the film. Suitable active ingredients may also be swallowed after dissolving the oral thin film in the mouth and thus be taken up via the gastrointestinal tract.

PCT Patent Application Publication number 2011/134846 A1 discloses multilayer oral thin films comprising an active substance-containing layer. U.S. Pat. Publication No. 2013/0017235 A1 discloses multilayer oral thin films in which an active substance-containing layer is enclosed by two water-swellable polymer layers. PCT Patent Application Publication number WO2009052421 A1 discloses film compositions comprising an active ingredient and a coating on the said film layer to provide the delivery of the active ingredient at the required rate.

U.S. Pat. No. 9,050,307 discloses a method of preparation and composition of a levothyroxine in aqueous solvent for oral administration. U.S. Pat. No. 7,723,390 discloses swallowable uniform soft-gel matrix comprising thyroid hormones. PCT Patent Application Publication number WO2018007466 Al discloses method of preparation of levothyroxine oral solution in a water-miscible organic solvent or a sugar alcohol. PCT Patent Application Publication number WO2007077252 A1 discloses liquid pharmaceutical composition.

None of the prior arts discloses a levothyroxine formulation for oral administration, which can be administered without any water and which does not require swallowing of a tablet or a significant volume of solution, which has a small size, thus being more likely to be available when needed, which has a fast systemic absorption, and which can be administered also to patients in distress or even unconscious patients.

The inventors of this invention have been surprisingly able to design levothyroxine oral film composition using film forming polymer, and a plasticizer with or without preservative, sweetner and other auxillary film forming agents.

### SUMMARY OF THE INVENTION

The present invention provides oral film dosage forms that are formulated or administered for gastrointestinal absorption of the active pharmaceutical agent. These oral films are mouth dissolving or soluble film, non-mucoadhesive film, fast disintegrating film; they quickly disintegrate in the mouth when exposed to saliva; and they are absorbed predominantly through the gastrointestinal tract.

In one general aspect, there is provided a pharmaceutical composition in the form of an oral film, wherein each oral film comprises - (i) levothyroxine or its salt, (ii) a film forming polymer, and (iii) a plasticizer.

In another aspect, each oral film comprises the active drug Levothyroxine or its salt in the range of about 10 mcg to about 300 mcg.

In another aspect, the oral film comprises levothyroxine or its salt in the range of about 0.001% w/w to about 0.75% w/w based on the total weight of the film.

In another aspect, the oral film further comprises a pharmaceutically acceptable excipient or any other suitable and required auxiliary fil forming excipients.

In another aspect, there is provided a pharmaceutical composition in the form of an oral film, wherein each oral film comprises - (i) levothyroxine or its salt, (ii) a film forming polymer, (iii) a plasticizer, and (iv) a pharmaceutically acceptable excipient or any other suitable and required auxiliary fil forming excipients.

In another aspect, there is provided a pharmaceutical composition in the form of an oral film, for administration into buccal cavity, comprises - (i) levothyroxine or its salt in the range of about 0.001% w/w to about 0.75% w/w, (ii) a film forming polymer, (iii) a plasticizer, and optionally, (iv) a pharmaceutically acceptable excipient or any other suitable and required auxiliary fil forming excipients, wherein the weight ratio of the plasticizer to the film forming polymer is in the range of about 1:1 to 1:10.

In another aspect, the film forming polymer is selected from one or more of cellulose derivatives; polyhydric alcohols; saccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; fat; wax; fatty acid; fatty acid ester; long chain monohydric alcohol or their ester; or mixtures thereof.

In another aspect, the film forming polymer is selected from one or more of cellulose derivatives; polyhydric alcohols; saccharides, polysaccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; fat; wax; fatty acid; fatty acid ester; long chain monohydric alcohol or their ester; or mixtures thereof.

In another aspect, the plasticizer is selected from, polyethylene glycol (PEG), PEG 400, propylene glycol, glycerol, triethyl citrate and polysorbate.

In another aspect, the film forming polymer is selected from one or more of (a) the cellulosic derivatives are selected from one or more of ethyl cellulose, methylcellulose, hydroxypropylmethylcellulose (HPMC), Methocel E15, Methocel K15, hydroxypropyl cellulose (HPC), hydroxyethyl cellulose, hydroxymethyl cellulose, hydroxypropyl ethylcellulose, carboxymethylethyl cellulose, carboxyethylcellulose, carboxymethyl hydroxyethylcellulose, hydroxyethylmethyl carboxymethyl cellulose, hydroxyethyl methyl cellulose, carboxymethyl cellulose (CMC), methylhydroxyethyl cellulose, methylhydroxypropyl cellulose, carboxymethyl sulfoethyl cellulose, sodium carboxymethyl cellulose, and combinations thereof, (b) polysaccharides such as Sodium alginate and like, (c) gums such as Xanthan gum and like, and (d) polymers such as Eudragit EPO.

In another aspect, the weight ratio of the plasticizer to the film forming polymer is in the range of about 1:3 to about 1:6.

In another aspect, the pharmaceutically acceptable excipient includes the pharmaceutically acceptable excipient includes an aqueous solvent, organic solvent, a base, a buffer, a sweetener, a colour additive, a flavouring agent, a preservative or mixtures thereof.

In another aspect, the pharmaceutically acceptable excipient is selected from one or more of (a) organic solvent, selected from the group of acetone, alcohol, ethanol, benzyl alcohol, benzyl benzoate, propylene glycol, butylene glycol, dibutyl phthalate, diethyl phthalate, dimethyl phthalate, dimethyl sulfoxide, dimethylacetamide, glycofurol, glycerin, isopropyl alcohol, isopropyl myristate, isopropyl palmitate, polyethylene glycol, propylene carbonate, pyrrolidone or mixtures thereof, (b) buffer is selected from the group of citric acid monohydrate, citrate, phosphate, succinate, tartrate, fumarate, gluconate, oxalate, lactate, acetate, histidine, sodium hydroxide or mixtures thereof, (c) the sweetener is selected from sucrose, mannitol, sucralose, aspartame and acesulfame, (d) the flavouring agent is selected from menthol, citric acid, lecithin, vanilla essence, peppermint essence and apple essence, (e) the preservative is selected from the group of sodium methyl hydroxyl benzoate, propyl hydroxyl benzoate, sorbic acid, paraben, bronopol, imidurea, phenoxyethanol, phenylmercuric acetate, benzyl alcohol, phenylmercuric borate, chlorocresol, benzethonium chloride, phenylethyl alcohol, benzalkonium chloride, hexetidine, chlorobutanol, cresol, cetylpyridinium chloride, phenylmercuric nitrate, chloroxylenol, propionic acid, phenol, thimerosal, sulfur dioxide, boric acid, edetic acid, sodium propionate, calcium chloride, sodium acetate, sodium sulfite, monothioglycerol, cetrimide, calcium acetate, butylene glycol, sodium metabisulfite, alcohol, propyl gallate, potassium metabisulfite, sodium lactate, chlorhexidine, calcium lactate, pentetic acid, propylene glycol alginate, sodium borate, magnesium trisilicate, isopropyl alcohol, dimethyl ether, butylated hydroxyanisole, pyrrolidone, lactic acid, dimethyl sulfoxide, or mixtures thereof, and (f) the colour additives are FD &C red # 40 and like.

In another aspect, there is provided a process for preparing the pharmaceutical composition of levothyroxine or its salt in the form of oral film, the process comprising the steps of:
a) Add weighed quantity of Levothyroxine sodium to vehicle phase/solvents and mix for 15-30 min to get clear solution.
b) Add weighed quantity of polymers to step (a) solution and mix for 30-45 min.
c) Add weighed quantity of plasticizer and other excipients to step (b) solution and mix for 15-20 min.
d) Observe Step (c) solution for clarity and cast on plain glass surface.
e) Dry Step (d) casted solution in an oven at 30-40° C. to forms non-sticky films and pack properly

In another aspect, there is provided a process for preparing the pharmaceutical composition of levothyroxine or its salt in the form of oral film, the process comprising the steps of:
a) adding weighed quantity of levothyroxine or its salt to solvent or mix of solvents and mixing for 15-30 min to get clear solution.
b) adding weighed quantity of polymers to step (a)′solution and mixing for 30-45 min. alternatively, polymer can be dissolved and dispersed separately in another portion of solvent mix.
c) adding weighed quantity of plasticizer and other pharmaceutically acceptable excipients to step (b) solution and mixing for 15-20 min.
d) spraying of the solution using roll or slot-die coating process followed by drying, lamination, and pouch packaging.
e) dry and/or wet bond lamination, and curing of the films can be done as required and deemed necessary.
f) drying of the prepared films can be done by roll support, Belt and/or air flotation methods.
g) alternatively, lab scale preparation can be prepared by casting of Step (c) solution on plain glass surface.
h) drying of Step(d) casted solution in an oven at 30° C.-70° C. to form non-sticky films and finally packing it into a pouch, to obtain a pharmaceutical composition of levothyroxine or its salt in the form of oral film.

In another aspect, the oral film can be of various types such as and including a mouth dissolving or soluble film, non-mucoadhesive film, fast disintegrating film, buccal film, mucoadhesive film, sublingual film, edible film or wafer.

In another aspect, the oral film has surface area in the range of about 0.5 cm² to about 50 cm².

In another aspect, the oral film has a thickness of about 0.5 mm to about 5 mm.

In another aspect, there is provided a pharmaceutical composition in the form of buccal film, wherein each buccal film comprises: (a) Levothyroxine Sodium, (b) Methocel E15, (c) Propylene glycol, (d) FD &C red # 40, (e) Peppermint flavour, (f) Ethanol, and (g) Purified water.

In another aspect, there is provided a pharmaceutical composition in the form of buccal film, wherein each buccal film comprises: (a) Levothyroxine Sodium, (b) Sodium alginate, (c) Glycerin, (d) FD &C red # 40, (e) Peppermint flavour, (f) Ethanol, and (g) Purified water.

In another aspect, there is provided a pharmaceutical composition in the form of sublingual film, wherein each sublingual film comprises: (a) Levothyroxine Sodium, (b) Sodium carboxymethyl cellulose, (c) Propylene glycol, (d) FD &C red # 40, (e) Peppermint flavour, (f) Ethanol, and (g) Purified water.

In another aspect, there is provided a pharmaceutical composition in the form of sublingual film, wherein each sublingual film comprises: (a) Levothyroxine Sodium, (b) Hydroxyethyl cellulose, (c) PEG 400, (d) FD &C red # 40, (e) Peppermint flavour, (f) Ethanol, and (g) Purified water.

In another aspect, there is provided a pharmaceutical composition in the form of mucoadhesive film, wherein each mucoadhesive film comprises: (a) Levothyroxine Sodium, (b) Methocel k15, (c) Propylene glycol, (d) FD &C red # 40, (e) Peppermint flavour, (f) Ethanol, and (g) Purified water.

In another aspect, there is provided a pharmaceutical composition in the form of mucoadhesive film, wherein each mucoadhesive film comprises: (a) Levothyroxine Sodium, (b) Sodium carboxymethyl cellulose, (c) Povidone K 30, (d) Glycerin (e) FD &C red # 40, (f) Peppermint flavour, and (g) Ethanol.

In another aspect, the compositions according to the invention comprise active drug potency (assay) preferably between 95%- 105% even after environmental exposure of one or more of the following:
(i) storage for one to three months at a temperature of 40° C. (±2° C.) and a relative humidity of 75% (± 5 %); and
(ii) storage for three to twelve months at a temperature of 25° C. (±2° C.) and a relative humidity of 65% (± 5 %).

In another aspect, the compositions according to the invention comprise comprising less than about 2% total impurities following one or more of the following:
(i) storage for one to three months at a temperature of 40° C. (±2° C.) and a relative humidity of 75% (± 5 %); and
(ii) storage for three to twelve months at a temperature of 25° C. (±2° C.) and a relative humidity of 65% (± 5 %).

In another aspect, the compositions according to the invention comprise disintegration not more than 1 minute following one or more of the following:
(i) storage for one to three months at a temperature of 40° C. (±2° C.) and a relative humidity of 75% (± 5 %); and
(ii) storage for three to twelve months at a temperature of 25° C. (±2° C.) and a relative humidity of 65% (± 5 %).

In one general aspect, there is provided a pharmaceutical composition in the form of an oral film, wherein each oral film comprises - (i) about 10 mcg to about 300 mcg of levothyroxine or its salt, (ii) a film forming polymer, and (iii) a plasticizer.

In another aspect, there is provided a method of treating hypothyroidism and/or pituitary thyrotrophic suppression in a subject, said method comprising orally administering to the subject therapeutically effective amount of a pharmaceutical composition in the form of an oral film, comprising levothyroxine or its salt in the range of about 0.001% w/w to about 0.75% w/w.

In another aspect, there is provided a pharmaceutical composition in the form of oral film comprising levothyroxine; wherein the film is designed to be applied on to the top or under the tongue and dissolve and get absorbed from there in 4 to 60 seconds and then is swallowed with saliva.

In another aspect, there is provided a pharmaceutical composition in the form of mouth dissolving film comprising levothyroxine; wherein the film is designed to be applied on to the top or under the tongue and dissolve and get absorbed from there in about 4 to about 60 seconds and then is swallowed with saliva.

In another aspect, there is provided a pharmaceutical composition in the form of oral film comprising levothyroxine; wherein the film is packed in individual foil-foil sealed child resistant pouch.

The details of one or more embodiments of the invention are set forth in the description below. Other features, objects and advantages of the invention will be apparent from the following description, including claims.

### DETAILED DESCRIPTION OF THE INVENTION

The oral film of the present invention and the methods of using the films are characterized by a number of features that ensure their bioequivalence to a comparable immediate release tablet or capsule or orally dissolving/dispersing tablet (ODT), including: the films may be engineered or used so that the active pharmaceutical agent is swallowed and absorbed predominantly or entirely through the gastrointestinal tract, instead of being absorbed through the oral mucosa; if necessary, the films or active pharmaceutical agents may be formulated so that absorption of active pharmaceutical agent through the oral mucosa is retarded; the films are typically designed for rapid disintegration when taken orally, and are most often swallowed in less than thirty or sixty seconds after administration; the films are usually applied directly onto the tongue to promote mixing with the saliva and subsequent swallowing of the active ingredient, and thereby discourage mucosal absorption; and water could be additionally swallowed within about thirty or sixty seconds after administration of the film, to further promote swallowing of the active agent and gastrointestinal absorption.

The term “non-mucoadhesive” means that the dosage form is not designed for administration of the active pharmaceutical agent through the oral mucosa. I.e. the dosage form is not designed to adhere to the mucosal surfaces of the buccal cavity as an intact film or disintegrated film residue.

The term “pharmaceutical composition” or “pharmaceutical formulation” or pharmaceutical dosage form” can be used interchangeably and refers to the combination of one or more active ingredients and one or more excipients.

As used herein, “disintegrate” or “disintegrating” means the process whereby an oral dosage form falls apart into smaller aggregates or particles.

As used herein, “dissolve” or “dissolving” means the process whereby a solid becomes incorporated into a liquid so as to form a solution.

An “orally dissolving or orally dispersible tablet” (“ODT”) refers to an uncoated tablet intended to be placed in the mouth where it can disperse rapidly before being swallowed .An ODT disintegrates within three minutes when tested according to the disintegration testing as described in European Pharmacopoeia .

“METHOCEL E15” is a low molecular weight hydroxypropyl methylcellulose (HPMC) thickener.

“FD&C Red No. 40” refers to the water soluble azo dye disodium salt of 6-hydroxy-5-[(2-methoxy-5-methyl-4-sulfophenyl)azo]-2-naphthalenesulfonic acid. FD&C Red No. 40 has an orange-red hue and is approved by the FDA for use in food, drugs, and cosmetics according to the specifications set forth by the FDA.1,2. FD&C Red No. 40, also known as Allura Red AC, is commonly used in liquid medications with cherry, strawberry or “berry” flavoring. It may also be used as a colorant in tablets and capsules. The FD&C notation specifies the color is approved for use in foods, drugs and cosmetics.

“Sodium alginate” refers to the sodium salt of alginic acid, a natural polysaccharide found in brown algae. It is generally used as a stabilizer and thickener in the food industry

The term ‘pharmaceutically acceptable excipient’ according to the present invention means, but not limited to, any inactive ingredient which is required for the formulation of oral film according to present invention. Particularly the excipient includes, but not limited to, lubricants, buffering agents, stabilizers, pigments, coloring agents, fillers, bulking agents, sweetening agents, flavoring aids, fragrances, release modifiers, adjuvants, plasticizers, granulating agents, diluents, binders, disintegrating agents, humectants, buffers, absorbents, glidants, anti-foaming agents, adhesives, anti-adherents, acidulants, softeners, resins, demulcents, solvents, surfactants, emulsifiers, elastomers, release agents, extenders, antiblocking agents, antitacking agents in amounts suitable for their intended purpose.

The flavoring agents that can be used include those known to the skilled artisan, such as natural and artificial flavors. These flavoring agent may be chosen from synthetic flavor oils and flavoring aromatics, and/or oils, oleo resins and extracts derived from plants, leaves, flowers, fruits and so forth, and combinations thereof. Representative flavor oils include: spearmint oil, cinnamon oil, peppermint oil, clove oil, bay oil, thyme oil.

Also useful are artificial, natural or synthetic flavors such as vanilla, chocolate, coffee, cocoa and fruit essences including apple, strawberry, raspberry, cherry, plum and so forth. These flavoring agents can be used individually or in admixture.

The term “buffer” refers to the component that improves isotonicity and chemical stability of the formulation, and functions to maintain suitable pH.

The term “organic solvent” refers to organic chemical compound that dissolves another to form a solution.

Oral films suitable for use in preparing the disclosed dosage forms are typically comprised of at least one water soluble polymer. In certain embodiments, the disintegrating film does not include insoluble polymers or other materials that can leave a gritty, unpleasant residue. Surfactants, polyalcohols, and or plasticizers may be incorporated into the disintegrating film to facilitate or enhance wettability and disintegration of the film. The film-forming polymer or combination of film-forming polymers can comprise 20% to 80% or 30% to 70% of the weight of the film oral dosage form on a dry basis.

The film-forming polymer according to the present invention provides a physiologically acceptable film and can be selected from the group consisting of pullulan, hydroxypropylmethyl cellulose, hydroxyethyl cellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof.

The base polymer for the film is “water-soluble polymer” which means polymers that dissolve or disperse in water to give a colloidal solution or dispersion at a temperature of less than 30° C. (for example from 10 to 20° C.). Generally the water-soluble polymers will have a solubility in water of at least 20 mg/ml, suitably at least 30 mg/ml at a temperature of 10 to 20° C. (wherein the solubility is determined in un-buffered distilled water). Suitable water-soluble polymers include but not limited to, those listed in the Handbook of Pharmaceutical Excipients, 3^{rd} Edition American Pharmaceutical Association, for example methylcellulose, hydroxyethylcellulose, hydroxypropylcellulose, hydroxypropyl methylcellulose, hydroxybutyl methylcellulose, hydroxyethyl ethylcellulose, a water-soluble salt of carboxymethylcellulose (for example sodium carboxymethylcellulose) and a water-soluble salt of carboxymethyl hydroxyethyl cellulose (for example sodium carboxymethyl hydroxyethylcellulose). More particularly suitable water-soluble polymer is selected from, for example, hydroxyethylcellulose, hydroxypropylcellulose, hydroxypropyl methylcellulose, and a water-soluble salt of carboxymethylcellulose (for example sodium carboxymethyl cellulose) etc.

The terms “surfactant” and “polyalcohol” are intended to have their ordinary meanings. Specifically, the term “surfactant” is intended to mean an amphophilic compound that lowers the surface tension of a liquid, the interfacial tension between two liquids, or the interfacial tension between a liquid and a solid. Examples of surfactants that can be used in a disintegrating film of an oral dosage form are known and include polyoxy-ethylene sorbitan fatty acid esters, an .alpha.-hydro-co-hydroxypoly (oxyethylene) poly (oxypropylene) poly(oxyethylene) block copolymer, a polyoxyethylene allyl ether, a polyoxyethylene or a castor oil derivative. Combinations of surfactants can be used. The term “polyalcohol” means a sugar alcohol, which is a hydrogenated form of a carbohydrate having a carbonyl group that has been reduced to a primary or secondary hydroxyl group. Polyalcohols are also distinguishable based on their chemical formula. Polyalcohols have the general formula H(HCHO)ₙ+1H, whereas sugars have the general formula H(HCHO)ₙ HCO. Common examples of polyalcohols or sugar alcohols that can be used from the disclosed films include glycol, glycerol, erythritol, threitol, arabitol, xylitol, ribitol, mannitol, sorbitol, galactitol, fucitol, iditol, inositol, volemitol, isomalt, maltitol, lactitol, maltotritol and maltotetraitol.

In another embodiment, there is provided a pharmaceutical composition, wherein the preservative is selected from the group of sodium methyl hydroxyl benzoate, propyl hydroxyl benzoate, sorbic acid, paraben, bronopol, imidurea, phenoxyethanol, phenylmercuric acetate, benzyl alcohol, phenylmercuric borate, chlorocresol, benzethonium chloride, phenylethyl alcohol, benzalkonium chloride, hexetidine, chlorobutanol, cresol, cetylpyridinium chloride, phenylmercuric nitrate, chloroxylenol, propionic acid, phenol, thimerosal, sulfur dioxide, boric acid, edetic acid, sodium propionate, calcium chloride, sodium acetate, sodium sulfite, monothioglycerol, cetrimide, calcium acetate, butylene glycol, sodium metabisulfite, alcohol, propyl gallate, potassium metabisulfite, sodium lactate, chlorhexidine, calcium lactate, pentetic acid, propylene glycol alginate, sodium borate, magnesium trisilicate, isopropyl alcohol, dimethyl ether, butylated hydroxyanisole, pyrrolidone, lactic acid, dimethyl sulfoxide, and mixtures thereof.

In another embodiment, the one or more alkalizer is selected from group comprising of calcium carbonate, calcium hydroxide, magnesium carbonate, magnesium hydroxide, magnesium silicate, magnesium aluminate or aluminum magnesium hydroxide, sodium hydroxide, sodium chloride, tromethamine.

In another embodiment, there is provided a pharmaceutical composition; wherein the one or more alkalizer comprises sodium hydroxide or combination thereof.

Sweetener provides sweetness and taste masking of pharmaceutical active(s) as well as some body and thickness. Sucrose, glucose or table sugar, often in liquid form, may be used. Alternatively, or additionally if greater sweetening is desired, sugar alcohols such as sorbitol, maltitol, and mannitol can be used to provide sweetness.

Examples of suitable flavoring agents include, but are not limited to, natural and artificial flavors such as mints (i.e., peppermint, etc.), menthol, chocolate, artificial chocolate, bubblegum, both artificial and natural fruit flavors (i.e., cherry, grape, orange, blue berry, strawberry, etc.) and combinations of two or more thereof. Flavoring agents are generally provided as a minor component of the composition in amounts effective to provide palatable flavor to the compositions.

Sweeteners, flavoring agents, and refreshing agents can be added in quantities, generally up to a total amount of about 5% to about 10% of the weight of the film on a dry basis, e.g., about 0.1% to about 10%, or about 0.5% to about 5%.

In order to promote adhesion of the levothyroxine oral film to oral mucosa, it is advantageous to add a mucoadhesive agent to the film product. Examples of mucoadhesive agents that can be added to the levothyroxine oral film to promote adhesion to oral mucosa include sodium alginate, sodium carboxymethyl cellulose, guar gum, hydroxyethyl cellulose, hydroxypropyl cellulose, hydroxypropylmethyl cellulose, karya gum, methylcellulose, polyethylene oxide, retene and tragacanth. Such mucoadhesive agent may be added to the film formulation in an amount of from about 0.5% to about 20%, or about 1% to about 5%, of the total weight of the film on a dry basis.

Plasticizers can be advantageously employed in the film formulations as needed to suitably modify the flexibility of the film to facilitate processing and allow the film to easily conform to the shape of the oral mucosa to which the film is applied. Plasticizers that can be effectively employed in the disclosed antihistamines film oral dosage forms to improve flexibility of the film include polyethylene glycol (PEG), polysorbate, ethylene glycol, propylene glycol, tributyl citrate, tri ethyl citrate and glycerol. Depending on the selected film-forming polymers and other components of the film formulation, a suitable amount of plasticizer is typically from about 0.1% to 10%, 0.5% to 5%, or 1% to 5%.

According to some embodiment, a method of forming an oral film of the present invention includes combining the various ingredients in generally any order, employing water, a combination of water and water-miscible solvents such as lower alcohols (e.g., ethanol) or organic solvents alone or as a mixture. For example, the plasticizer and additives (e.g., sweetening agents, colorants, flavoring agents, and opacifying agents) can be dissolved or dispersed in a sufficient amount of solvent that is agitated to form a homogenous solution or suspension to which the water soluble polymer(s) is (are) added. Heat, vacuum and agitation may be applied as needed during addition of the water soluble polymer until a homogenous solution or homogenous suspension is obtained. Thereafter, the active ingredient(s) is (are) added, and the solution or suspension is cast or coated onto a carrier material and dried to form a film. Examples of suitable carrier materials include non-siliconized polyethylene terephthalate film, non-siliconized kraft paper, polyethylene-impregnated kraft paper and non-siliconized polyethylene film. The liquid film composition can be coated onto the carrier material using generally any conventional coating equipment, including knife-over-roll, extrusion die, reverse roll, or Meyer roll coating equipment.

In one general aspect, there is provided a pharmaceutical composition in the form of an oral film, wherein each oral film comprises - (i) about 10 mcg to about 300 mcg of levothyroxine or its salt, (ii) a film forming polymer, and (iii) a plasticizer.

In another embodiment of this aspect, the oral film comprises about 25 mcg to about 100 mcg or about 110 mcg to about 150 mcg or about 170 mcg to about 200 mcg or about 200 mcg to about 300 mcg of levothyroxine or its salt.

In another embodiment, the oral film comprises 25 mcg or 50 mcg or 75 mcg or 88 mcg or 100 mcg or 112 mcg or 125 mcg or 137 mcg or 150 mcg or 175 mcg or 200 mcg or 300 mcg of levothyroxine or its salt.

In another aspect, the oral film can be of various types such as and including a mouth dissolving or soluble film, non-mucoadhesive film, fast disintegrating film, buccal film, mucoadhesive film, sublingual film, edible film or wafer.

In one general aspect, there is provided a pharmaceutical composition in the form of a mouth dissolving film, wherein each oral film comprises - (i) about 10 mcg to about 300 mcg of levothyroxine or its salt, (ii) a film forming polymer, and (iii) a plasticizer.

In one general aspect, there is provided a pharmaceutical composition in the form of a non-mucoadhesive film, wherein each oral film comprises - (i) about 10 mcg to about 300 mcg of levothyroxine or its salt, (ii) a film forming polymer, and (iii) a plasticizer.

In one general aspect, there is provided a pharmaceutical composition in the form of a mucoadhesive film, wherein each oral film comprises - (i) about 10 mcg to about 300 mcg of levothyroxine or its salt, (ii) a film forming polymer, and (iii) a plasticizer.

In one general aspect, there is provided a pharmaceutical composition in the form of a buccal film, wherein each oral film comprises - (i) about 10 mcg to about 300 mcg of levothyroxine or its salt, (ii) a film forming polymer, and (iii) a plasticizer.

In one general aspect, there is provided a pharmaceutical composition in the form of a sublingual film, wherein each oral film comprises - (i) about 10 mcg to about 300 mcg of levothyroxine or its salt, (ii) a film forming polymer, and (iii) a plasticizer.

In one general aspect, there is provided a pharmaceutical composition in the form of a mouth dissolving film, wherein each oral film comprises - (i) about 10 mcg to about 300 mcg of levothyroxine or its salt, (ii) a film forming polymer, (iii) a plasticizer, and (iv) a pharmaceutically acceptable excipient or any other suitable and required auxiliary fil forming excipients.

In one general aspect, there is provided a pharmaceutical composition in the form of a non-mucoadhesive film, wherein each oral film comprises - (i) about 10 mcg to about 300 mcg of levothyroxine or its salt, (ii) a film forming polymer, (iii) a plasticizer, and (iv) a pharmaceutically acceptable excipient or any other suitable and required auxiliary fil forming excipients.

In one general aspect, there is provided a pharmaceutical composition in the form of a mucoadhesive film, wherein each oral film comprises - (i) about 10 mcg to about 300 mcg of levothyroxine or its salt, (ii) a film forming polymer, (iii) a plasticizer, and (iv) a pharmaceutically acceptable excipient or any other suitable and required auxiliary fil forming excipients.

In one general aspect, there is provided a pharmaceutical composition in the form of a buccal film, wherein each oral film comprises - (i) about 10 mcg to about 300 mcg of levothyroxine or its salt, (ii) a film forming polymer, (iii) a plasticizer, and (iv) a pharmaceutically acceptable excipient or any other suitable and required auxiliary fil forming excipients.

In one general aspect, there is provided a pharmaceutical composition in the form of a sublingual film, wherein each oral film comprises - (i) about 10 mcg to about 300 mcg of levothyroxine or its salt, (ii) a film forming polymer, (iii) a plasticizer, and (iv) a pharmaceutically acceptable excipient or any other suitable and required auxiliary fil forming excipients.

In another embodiment, the oral film has surface area in the range of about 0.5 cm² to about 50 cm². In another embodiment, the oral film has a thickness of about 0.5 mm to about 5 mm. In another embodiment, the oral film has a thickness of about 1 mm to 5 mm. In another embodiment, the oral film has a thickness of about 5 mm to 10 mm. In another embodiment, the oral film has a thickness of about 10 mm to 20 mm. In another embodiment, the oral film comprises levothyroxine or its salt in the range of about 0.001% w/w to about 0.75% w/w based on the total weight of the film.

In yet another aspect, there is provided an oral film pharmaceutical composition for administration into oral cavity, comprising - (i) levothyroxine or its salt in the range of about 0.001% w/w to about 0.75% w/w, (ii) a film forming polymer, (iii) a plasticizer, and (iv) a pharmaceutically acceptable excipient or any other suitable and required auxiliary fil forming excipients, wherein the weight ratio of plasticizer to the film forming polymer is about 1:1 to about 1:10.

In an embodiment, the weight ratio of plasticizer to the film forming polymer is about 1:2 to about 1:8. In an embodiment, the weight ratio of the plasticizer to the film forming polymer is about 1:3 to about 1:6. In an embodiment, the weight ratio of the plasticizer to the film forming polymer is about 1:4 to about 1:5.

The term “related substances” as used herein refers to one or more impurities present in the pharmaceutical composition according to the invention. Such impurities may be present in the composition due to degradation of one or more components in the composition, for example the active or inactive ingredients. The amount of impurities is calculated on the basis of the levothyroxine or its salt present in the composition.

In some embodiments, there is provided a pharmaceutical composition in the form of an oral film, wherein each oral film comprises - (i) levothyroxine or its salt, (ii) a film forming polymer, and (iii) a plasticizer.

In some embodiments, each oral film comprises the active drug Levothyroxine or its salt in the range of about 10 mcg to about 300 mcg.

In some embodiments, the oral film comprises levothyroxine or its salt in the range of about 0.001% w/w to about 0.75% w/w based on the total weight of the film.

In some embodiments, the oral film further comprises a pharmaceutically acceptable excipient or any other suitable and required auxiliary fil forming excipients.

In some embodiments, there is provided a pharmaceutical composition in the form of an oral film, wherein each oral film comprises - (i) levothyroxine or its salt, (ii) a film forming polymer, (iii) a plasticizer, and (iv) a pharmaceutically acceptable excipient or any other suitable and required auxiliary fil forming excipients.

In some embodiments, there is provided a pharmaceutical composition in the form of an oral film, for administration into buccal cavity, comprises - (i) levothyroxine or its salt in the range of about 0.001% w/w to about 0.75% w/w, (ii) a film forming polymer, (iii) a plasticizer, and optionally, (iv) a pharmaceutically acceptable excipient, wherein the weight ratio of the plasticizer to the film forming polymer is in the range of about 1:1 to 1:10.

In some embodiments, the film forming polymer is selected from one or more of cellulose derivatives; polyhydric alcohols; saccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; fat; wax; fatty acid; fatty acid ester; long chain monohydric alcohol or their ester; or mixtures thereof.

In some embodiments, the film forming polymer is selected from one or more of cellulose derivatives; polyhydric alcohols; saccharides, polysaccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; fat; wax; fatty acid; fatty acid ester; long chain monohydric alcohol or their ester; or mixtures thereof.

In some embodiments, the plasticizer is selected from, polyethylene glycol (PEG), PEG 400, propylene glycol, glycerol, triethyl citrate and polysorbate.

In some embodiments, the film forming polymer is selected from one or more of (a) the cellulosic derivatives are selected from one or more of ethyl cellulose, methylcellulose, hydroxypropylmethylcellulose (HPMC), Methocel E15, Methocel K15, hydroxypropyl cellulose (HPC), hydroxyethyl cellulose, hydroxymethyl cellulose, hydroxypropyl ethylcellulose, carboxymethylethyl cellulose, carboxyethylcellulose, carboxymethyl hydroxyethylcellulose, hydroxyethylmethyl carboxymethyl cellulose, hydroxyethyl methyl cellulose, carboxymethyl cellulose (CMC), methylhydroxyethyl cellulose, methylhydroxypropyl cellulose, carboxymethyl sulfoethyl cellulose, sodium carboxymethyl cellulose, and combinations thereof, (b) polysaccharides such as Sodium alginate and like, (c) gums such as Xanthan gum and like, and (d) polymers such as Eudragit EPO.

In some embodiments, the weight ratio of the plasticizer to the film forming polymer is in the range of about 1:3 to about 1:6.

In some embodiments, the pharmaceutically acceptable excipient includes the pharmaceutically acceptable excipient or any other suitable and required auxiliary fil forming excipients includes an aqueous solvent, organic solvent, a base, a buffer, a sweetener, a colour additive, a flavouring agent, a preservative or mixtures thereof.

In some embodiments, the pharmaceutically acceptable excipient is selected from one or more of (a) organic solvent, selected from the group of acetone, alcohol, ethanol, benzyl alcohol, benzyl benzoate, propylene glycol, butylene glycol, dibutyl phthalate, diethyl phthalate, dimethyl phthalate, dimethyl sulfoxide, dimethylacetamide, glycofurol, glycerin, isopropyl alcohol, isopropyl myristate, isopropyl palmitate, polyethylene glycol, propylene carbonate, pyrrolidone or mixtures thereof, (b) buffer is selected from the group of citric acid monohydrate, citrate, phosphate, succinate, tartrate, fumarate, gluconate, oxalate, lactate, acetate, histidine, sodium hydroxide or mixtures thereof, (c) the sweetener is selected from sucrose, mannitol, sucralose, aspartame and acesulfame, (d) the flavouring agent is selected from menthol, citric acid, lecithin, vanilla essence, peppermint essence and apple essence, (e) the preservative is selected from the group of sodium methyl hydroxyl benzoate, propyl hydroxyl benzoate, sorbic acid, paraben, bronopol, imidurea, phenoxyethanol, phenylmercuric acetate, benzyl alcohol, phenylmercuric borate, chlorocresol, benzethonium chloride, phenylethyl alcohol, benzalkonium chloride, hexetidine, chlorobutanol, cresol, cetylpyridinium chloride, phenylmercuric nitrate, chloroxylenol, propionic acid, phenol, thimerosal, sulfur dioxide, boric acid, edetic acid, sodium propionate, calcium chloride, sodium acetate, sodium sulfite, monothioglycerol, cetrimide, calcium acetate, butylene glycol, sodium metabisulfite, alcohol, propyl gallate, potassium metabisulfite, sodium lactate, chlorhexidine, calcium lactate, pentetic acid, propylene glycol alginate, sodium borate, magnesium trisilicate, isopropyl alcohol, dimethyl ether, butylated hydroxyanisole, pyrrolidone, lactic acid, dimethyl sulfoxide, or mixtures thereof, and (f) the colour additives are FD &C red # 40 and like.

In some embodiments, there is provided a process for preparing the pharmaceutical composition of levothyroxine or its salt in the form of oral film, the process comprising the steps of:
a) add weighed quantity of Levothyroxine sodium to vehicle phase/solvents and mix for 15-30 min to get clear solution.
b) add weighed quantity of polymers to step (a) solution and mix for 30-45 min.
c) add weighed quantity of plasticizer and other excipients to step (b) solution and mix for 15-20 min.
d) observe Step (c) solution for clarity and cast on plain glass surface.
e) dry step (d) casted solution in an oven at 30-40° C. to forms non-sticky films and pack properly.

In some embodiments, there is provided a process for preparing the pharmaceutical composition of levothyroxine or its salt in the form of oral film, the process comprising the steps of:
a) adding weighed quantity of levothyroxine or its salt to solvent or mix of solvents and mixing for 15-30 min to get clear solution.
b) adding weighed quantity of polymers to step (a)′solution and mixing for 30-45 min. alternatively, polymer can be dissolved and dispersed separately in another portion of solvent mix.
c) adding weighed quantity of plasticizer and other pharmaceutically acceptable excipients to step (b) solution and mixing for 15-20 min.
d) spraying of the solution using roll or slot-die coating process followed by drying, lamination, and pouch packaging.
e) dry and/or wet bond lamination, and curing of the films can be done as required and deemed necessary.
f) drying of the prepared films can be done by roll support, Belt and/or air flotation methods.
g) alternatively, lab scale preparation can be prepared by casting of Step (c) solution on plain glass surface.
h) drying of Step(d) casted solution in an oven at 30° C.-70° C. to form non-sticky films and finally packing it into a pouch, to obtain a pharmaceutical composition of levothyroxine or its salt in the form of oral film.

In some embodiments, the oral film can be of various types such as and including a mouth dissolving or soluble film, non-mucoadhesive film, fast disintegrating film, buccal film, mucoadhesive film, sublingual film, edible film or wafer.

In some embodiments, the oral film has surface area in the range of about 0.5 cm² to about 50 cm².

In some embodiments, the oral film has a thickness of about 0.5 mm to about 5 mm.

In some embodiments, there is provided a pharmaceutical composition in the form of buccal film, wherein each buccal film comprises: (a) Levothyroxine Sodium, (b) Methocel E15, (c) Propylene glycol, (d) FD &C red # 40, (e) Peppermint flavour, (f) Ethanol, and (g) Purified water.

In some embodiments, there is provided a pharmaceutical composition in the form of buccal film, wherein each buccal film comprises: (a) Levothyroxine Sodium, (b) Sodium alginate, (c) Glycerin, (d) FD &C red # 40, (e) Peppermint flavour, (f) Ethanol, and (g) Purified water.

In some embodiments, there is provided a pharmaceutical composition in the form of sublingual film, wherein each sublingual film comprises: (a) Levothyroxine Sodium, (b) Sodium carboxymethyl cellulose, (c) Propylene glycol, (d) FD &C red # 40, (e) Peppermint flavour, (f) Ethanol, and (g) Purified water.

In some embodiments, there is provided a pharmaceutical composition in the form of sublingual film, wherein each sublingual film comprises: (a) Levothyroxine Sodium, (b) Hydroxyethyl cellulose, (c) PEG 400, (d) FD &C red # 40, (e) Peppermint flavour, (f) Ethanol, and (g) Purified water.

In some embodiments, there is provided a pharmaceutical composition in the form of mucoadhesive film, wherein each mucoadhesive film comprises: (a) Levothyroxine Sodium, (b) Methocel k15, (c) Propylene glycol, (d) FD &C red # 40, (e) Peppermint flavour, (f) Ethanol, and (g) Purified water.

In some embodiments, there is provided a pharmaceutical composition in the form of mucoadhesive film, wherein each mucoadhesive film comprises: (a) Levothyroxine Sodium, (b) Sodium carboxymethyl cellulose, (c) Povidone K 30, (d) Glycerin (e) FD &C red # 40, (f) Peppermint flavour, and (g) Ethanol.

In some embodiments, the compositions according to the invention comprise active drug potency (assay) preferably between 95%- 105% even after environmental exposure of one or more of the following:
(i) storage for one to three months at a temperature of 40° C. (±2° C.) and a relative humidity of 75% (± 5 %); and
(ii) storage for three to twelve months at a temperature of 25° C. (±2° C.) and a relative humidity of 65% (± 5 %).

In some embodiments, the compositions according to the invention comprise comprising less than about 2% total impurities following one or more of the following:
(i) storage for one to three months at a temperature of 40° C. (±2° C.) and a relative humidity of 75% (± 5 %); and
(ii) storage for three to twelve months at a temperature of 25° C. (±2° C.) and a relative humidity of 65% (± 5 %).

In some embodiments, the compositions according to the invention comprise disintegration not more than 1 minute following one or more of the following:
(i) storage for one to three months at a temperature of 40° C. (±2° C.) and a relative humidity of 75% (± 5 %); and
(ii) storage for three to twelve months at a temperature of 25° C. (±2° C.) and a relative humidity of 65% (± 5 %).

In some embodiments, there is provided a method of treating hypothyroidism and/or pituitary thyrotrophic suppression in a subject, said method comprising orally administering to the subject therapeutically effective amount of a pharmaceutical composition in the form of an oral film, comprising levothyroxine or its salt in the range of about 0.001% w/w to about 0.75% w/w.

In some embodiments, there is provided a pharmaceutical composition in the form of oral film comprising levothyroxine; wherein the film is designed to be applied on to the top or under the tongue and dissolve and get absorbed from there in 4 to 60 seconds and then is swallowed with saliva.

In some embodiments, there is provided a pharmaceutical composition in the form of mouth dissolving film comprising levothyroxine; wherein the film is designed to be applied on to the top or under the tongue and dissolve and get absorbed from there in about 4 to about 60 seconds and then is swallowed with saliva.

In some embodiments, there is provided a pharmaceutical composition in the form of oral film comprising levothyroxine; wherein the film is packed in individual foil-foil sealed child resistant pouch.

Advantageously, the compositions according to the invention are stable on storage, as assessed from the impurity content following storage at various conditions.

The invention is further illustrated by the following examples which are provided merely to be exemplary of the invention and do not limit the scope of the invention. Certain modifications and equivalents will be apparent to those skilled in the art and are intended to be included within the scope of the invention.

### EXAMPLES

The following examples illustrate the embodiments of the invention that are presently best known. However, it is to be understood that the following are only exemplary or illustrative of the application of the principles of the present invention. Numerous modifications and alternative compositions, methods and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

Table 1 and Table 2 provides the pharmaceutical compositions according to the invention.

TABLE 1

EXAMPLE 1: Levothyroxine sodium oral film composition
Examples

| Sr. | Ingredients | Quantity per film in mg | | | | | | | |
|---|---|---|---|---|---|---|---|---|---|
| | | 1A | 1B | 1C | 1D | 1E | 1F | 1G | 1H |
| 1 | Levothyroxine sodium | 0.05 | 0.15 | 0.025 | 0.25 | 0.05 | 0.05 | 0.05 | 0.05 |
| 2 | Eudragit EPO | 50 | - | - | - | - | - | - | - |
| 3 | Hypromellose E50 | - | 200 | - | - | - | - | - | - |
| 4 | Methocel E15 | - | - | 200 | 200 | - | - | 200 | - |
| 5 | Hydroxy ethyl cellulose | - | - | - | - | 150 | - | - | - |
| 6 | Carbopol | - | - | - | - | - | - | - | 175 |
| 7 | Xanthan gum | - | - | - | - | 175 | - | - | - |
| 8 | Triethyl citrate | 15 | - | - | - | - | - | - | - |
| 9 | PEG 400 | 15 | 40 | - | 40 | - | 40 | - | - |
| 10 | Propylene glycol | - | - | 40 | - | 40 | - | - | 40 |
| 11 | Glycerine | - | - | - | - | - | - | 40 | - |
| 12 | Isopropyl alcohol | q.s. | - | - | - | - | - | - | - |
| 13 | Sodium Hydroxide | - | 0.61 | 0.61 | 0.61 | 0.61 | 0.61 | 0.61 | 0.61 |
| 14 | FD &C red # 40 | - | - | 0.05 | 0.05 | 0.05 | 0.05 | 0.5 | 0.05 |
| 15 | Menthol | - | - | - | - | 0.5 | 0.5 | 0.5 | 0.5 |
| 16 | Ethanol | - | q.s. | q.s. | q.s. | q.s. | q.s. | q.s. | q.s. |
| 17 | Purified Water | - | q.s. | q.s. | q.s. | q.s. | q.s. | q.s. | q.s. |

q.s. = quantity sufficient

Manufacturing Process:
a) Add weighed quantity of Levothyroxine sodium to vehicle phase/solvents and mix for 15-30 min to get clear solution.
b) Add weighed quantity of polymers to step (a) solution and mix for 30-45 min.
c) Add weighed quantity of plasticizer and other excipients to step (b) solution and mix for 15-20 min.
d) Observe Step (c) solution for clarity and cast on plain glass surface.
e) Dry Step (d) casted solution in an oven at 30-40° C. to forms non-sticky films and pack properly

TABLE 2

EXAMPLE 2, 3, 4: Levothyroxine sodium oral film composition

| | | Example 2 Buccal Films | | Example 3 Sublingual films | | Example 4 Mucoadhesive films | |
|---|---|---|---|---|---|---|---|
| Sr. | Ingredients | Quantity per film in mg | | | | | |
| | | 2A | 2B | 3A | 3B | 4A | 4B |
| 1 | Levothyroxine Sodium | 0.05 | 0.05 | 0.05 | 0.05 | 0.05 | 0.05 |
| 2 | Methocel E15 | 150 | - | - | - | 60 | - |
| 3 | Sodium carboxymethyl cellulose | - | - | 50 | - | - | 40 |
| 4 | Hydroxy ethyl cellulose | - | - | - | 60 | - | - |
| 5 | PEG 400 | - | - | - | 30 | - | - |
| 6 | Propylene glycol | 40 | - | 20 | - | 30 | - |
| 7 | Povidone K 30 | - | - | - | - | - | 15 |
| 8 | Sodium alginate | - | 60 | - | - | - | - |
| 9 | Glycerin | - | 30 | - | - | - | 25 |
| 10 | FD &C red # 40 | 0.05 | 0.05 | 0.05 | 0.05 | 0.05 | 0.05 |
| 11 | Peppermint flavour | 0.05 | 0.05 | 0.05 | 0.05 | 0.05 | 0.05 |
| 12 | Ethanol | q.s. | q.s. | q.s. | q.s. | q.s. | q.s. |
| 13 | Purified water | q.s. | q.s. | q.s. | q.s. | q.s. | q.s. |

q.s. = quantity sufficient

Manufacturing Process: The process comprising the steps of:
a. adding weighed quantity of levothyroxine or its salt to solvent or mix of solvents and mixing for 15-30 min to get clear solution
b. adding weighed quantity of polymers to step (a)′solution and mixing for 30-45 min. alternatively, polymer can be dissolved and dispersed separately in another portion of solvent mix.
c. adding weighed quantity of plasticizer and other pharmaceutically acceptable excipients to step (b) solution and mixing for 15-20 min.
d. spraying of the solution using roll or slot-die coating process followed by drying, lamination, and pouch packaging.
e. dry and/or wet bond lamination, and curing of the films can be done as required and deemed necessary.
f. drying of the prepared films can be done by roll support, Belt and/or air flotation methods.
g. alternatively, lab scale preparation can be prepared by casting of Step (c) solution on plain glass surface.
h. drying of Step(d) casted solution in an oven at 30° C.-70° C. to form non-sticky films and finally packing it into a pouch, to obtain a pharmaceutical composition of levothyroxine or its salt in the form of oral film.

The compositions according to invention were also tested for stability up to three to twelve months at various conditions: (a) 40° C. ± 2° C. temperature and 75% RH ± 5% relative humidity; and (b) 25° C. ± 2° C. temperature and 65% RH ± 5% relative humidity. The results of the stability studies are provided in Tables 3 to 8.

TABLE 3

Stability- Buccal films Example 2A

| Test | Limit | Initial | 40° C. ± 2° C. / 75% RH ± 5% RH | | | 25° C. ± 2° C. / 65% RH ± 5% RH | | | |
|---|---|---|---|---|---|---|---|---|---|
| | | | 1 M | 2 M | 3 M | 3 M | 6 M | 9 M | 12 M |
| Assay | 95 - 105 % | 99.8 | 97.2 | 96.9 | 97.8 | 97.9 | 97.5 | 98.0 | 97.6 |
| Related Substance | Levothyroxine sodium - NMT 2.0% | 0.3 | 0.3 | 0.3 | 0.4 | 0.3 | 0.3 | 0.3 | 0.3 |
| | Any individual specified impurity - NMT 1.0% | 0.1 | 0.1 | 0.2 | 0.2 | 0.1 | 0.1 | 0.1 | 0.1 |
| | Any unspecified impurity - NMT 1.0% | 0.1 | 0.1 | 0.1 | 0.1 | 0.1 | 0.1 | 0.1 | 0.1 |
| | Total impurities - NMT 5.0% | 1.0 | 1.0 | 1.2 | 1.3 | 0.9 | 1.0 | 0.7 | 0.9 |
| Disintegration ( 25 ml 6.8 Phosphate buffer in petri dish ) | Not more than 1 minute | 45 Secs | 42 Secs | 43 Secs | 40 Secs | 52 Secs | 44 Secs | 49 Secs | 54 Secs |

TABLE 4

Stability- Buccal films Example 2B

| Test | Limit | Initial | 40° C. ± 2° C. / 75% RH ± 5% RH | | | 25° C. ± 2° C. / 65% RH ± 5% RH | | | |
|---|---|---|---|---|---|---|---|---|---|
| | | | 1 M | 2 M | 3 M | 3 M | 6 M | 9 M | 12 M |
| Assay | 95 - 105 % | 98.0 | 96.9 | 97.5 | 97.7 | 96.8 | 97.4 | 98.0 | 97.2 |
| Related Substance | Levothyroxine sodium - NMT 2.0% | 0.2 | 0.3 | 0.3 | 0.3 | 0.2 | 0.2 | 0.2 | 0.2 |
| | Any individual specified impurity - NMT 1.0% | 0.1 | 0.1 | 0.2 | 0.2 | 0.1 | 0.1 | 0.1 | 0.1 |
| | Any unspecified impurity - NMT 1.0% | 0.1 | 0.1 | 0.1 | 0.1 | 0.1 | 0.1 | 0.1 | 0.1 |
| | Total impurities - NMT 5.0% | 0.8 | 1.1 | 1.3 | 1.2 | 0.8 | 0.9 | 1.0 | 0.8 |
| Disintegration ( 25 ml 6.8 Phosphate buffer in petri dish ) | Not more than 1 minute | 30 Secs | 29 Secs | 25 Secs | 27 Secs | 32 Secs | 29 Secs | 30 Secs | 32 Secs |

TABLE 5

Stability- Sublingual films Example 3A

| Test | Limit | Initial | 40° C. ± 2° C. / 75% RH ± 5% RH | | | 25° C. ± 2° C. / 65% RH ± 5% RH | | | |
|---|---|---|---|---|---|---|---|---|---|
| | | | 1 M | 2 M | 3 M | 3 M | 6 M | 9 M | 12 |
| Assay | 95 - 105 % | 100.4 | 99.8 | 98.9 | 99.4 | 100.1 | 99.9 | 100.1 | 100.4 |
| Related Substance | Levothyroxine sodium - NMT 2.0% | 0.3 | 0.3 | 0.3 | 0.3 | 0.3 | 0.3 | 0.3 | 0.3 |
| | Any individual specified impurity - NMT 1.0% | 0.2 | 0.2 | 0.2 | 0.2 | 0.2 | 0.2 | 0.2 | 0.2 |
| | Any unspecified impurity - NMT 1.0% | 0.1 | 0.1 | 0.1 | 0.1 | 0.1 | 0.1 | 0.1 | 0.1 |
| | Total impurities - NMT 5.0% | 1.4 | 1.2 | 1.0 | 1.3 | 1.2 | 1.3 | 1.1 | 1.3 |
| Disintegration ( 25 ml 6.8 Phosphate buffer in petri dish ) | Not more than 1 minute | 25 Secs | 30 Secs | 24 Secs | 26 Secs | 28 Secs | 25 Secs | 29 Secs | 26 Secs |

TABLE 6

Stability- Sublingual films Example 3B

| Test | Limit | Initial | 40° C. ± 2° C. / 75% RH ± 5% RH | | | 25° C. ± 2° C. / 65% RH ± 5% RH | | | |
|---|---|---|---|---|---|---|---|---|---|
| | | | 1 M | 2 M | 3 M | 3 M | 6 M | 9 M | 12 |
| Assay | 95 - 105 % | 99.8 | 99.2 | 97.6 | 98.5 | 99.3 | 100.1 | 99.6 | 99.84 |
| Related Substance | Levothyroxine sodium - NMT 2.0% | 0.3 | 0.3 | 0.3 | 0.3 | 0.3 | 0.3 | 0.3 | 0.3 |
| | Any individual specified impurity - NMT 1.0% | 0.2 | 0.2 | 0.2 | 0.2 | 0.2 | 0.2 | 0.1 | 0.2 |
| | Any unspecified impurity - NMT 1.0% | 0.1 | 0.1 | 0.1 | 0.1 | 0.1 | 0.1 | 0.1 | 0.1 |
| | Total impurities - NMT 5.0% | 1.2 | 1.3 | 1.0 | 1.2 | 0.9 | 1.0 | 1.2 | 1.0 |
| Disintegration ( 25 ml 6.8 Phosphate buffer in petri dish ) | Not more than 1 minute | 43 Secs | 40 Secs | 45 Secs | 36 Secs | 42 Secs | 44 Secs | 41 Secs | 42 Secs |

TABLE 7

Stability- Mucoadhesive films Example 4A

| Test | Limit | Initial | 40° C. ± 2° C. / 75% RH ± 5% RH | | | 25° C. ± 2° C. / 65% RH ± 5% RH | | | |
|---|---|---|---|---|---|---|---|---|---|
| | | | 1 M | 2 M | 3 M | 3 M | 6 M | 9 M | 12 M |
| Assay | 95 - 105 % | 99.8 | 99.2 | 97.5 | 99.4 | 99.6 | 98.6 | 99.4 | 99.0 |
| Related Substance | Levothyroxine sodium - NMT 2.0% | 0.3 | 0.3 | 0.3 | 0.3 | 0.3 | 0.3 | 0.3 | 0.3 |
| | Any individual specified impurity - NMT 1.0% | 0.2 | 0.2 | 0.2 | 0.2 | 0.2 | 0.2 | 0.2 | 0.2 |
| | Any unspecified impurity - NMT 1.0% | 0.1 | 0.1 | 0.1 | 0.2 | 0.1 | 0.1 | 0.1 | 0.1 |
| | Total impurities - NMT 5.0% | 1.1 | 1.3 | 1.0 | 1.4 | 0.9 | 1.2 | 1.1 | 0.9 |
| Disintegration ( 25 ml 6.8 Phosphate buffer in petri dish ) | Not more than 1 minute | 54 Secs | 53 Secs | 49 Secs | 55 Secs | 51 Secs | 53 Secs | 56 Secs | 50 Secs |

TABLE 8

Stability- Mucoadhesive films Example 4B

| Test | Limit | Initial | 40° C. ± 2° C. / 75% RH ± 5% RH | | | 25° C. ± 2° C. / 65% RH ± 5% RH | | | |
|---|---|---|---|---|---|---|---|---|---|
| | | | 1 M | 2 M | 3 M | 3 M | 6 M | 9 M | 12 M |
| Assay | 95 - 105 % | 100.6 | 100.0 | 98.3 | 99.8 | 100.8 | 100.1 | 99.6 | 99.5 |
| Related Substance | Levothyroxine sodium - NMT 2.0% | 0.2 | 0.2 | 0.3 | 0.2 | 0.2 | 0.2 | 0.2 | 0.2 |
| | Any individual specified impurity - NMT 1.0% | 0.2 | 0.2 | 0.2 | 0.2 | 0.2 | 0.2 | 0.1 | 0.2 |
| | Any unspecified impurity - NMT 1.0% | 0.1 | 0.1 | 0.1 | 0.1 | 0.1 | 0.1 | 0.1 | 0.1 |
| | Total impurities - NMT 5.0% | 0.9 | 1.0 | 1.2 | 1.1 | 0.8 | 1.1 | 1.1 | 0.9 |
| Disintegration ( 25 ml 6.8 Phosphate buffer in petri dish ) | Not more than 1 minute | 23 Secs | 24 Secs | 29 Secs | 24 Secs | 26 Secs | 28 Secs | 30 Secs | 27 Secs |

We claim:

1. A pharmaceutical composition in the form of an oral film, wherein each oral film comprises - (i) levothyroxine or its salt, (ii) a film forming polymer, and (iii) a plasticizer.

2. The pharmaceutical composition according to claim 1, wherein each oral film comprises the active drug Levothyroxine or its salt in the range of about 10 mcg to about 300 mcg.

3. The pharmaceutical composition according to claim 1, wherein the oral film comprises levothyroxine or its salt in the range of about 0.001% w/w to about 0.75% w/w based on the total weight of the film.

4. The pharmaceutical composition according to claim 1, wherein each oral film further comprises a pharmaceutically acceptable excipient or any other suitable and required auxiliary fil forming excipients.

5. A pharmaceutical composition in the form of an oral film, for administration into buccal cavity, comprises - (i) levothyroxine or its salt in the range of about 0.001% w/w to about 0.75% w/w, (ii) a film forming polymer, (iii) a plasticizer, and optionally, (iv) a pharmaceutically acceptable excipient or any other suitable and required auxiliary fil forming excipients, wherein the weight ratio of the plasticizer to the film forming polymer is in the range of about 1:1 to 1:10.

6. The pharmaceutical composition according to any one of claims 1 to 5, wherein the film forming polymer is selected from one or more of cellulose derivatives; polyhydric alcohols; saccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; fat; wax; fatty acid; fatty acid ester; long chain monohydric alcohol or their ester; or mixtures thereof.

7. The pharmaceutical composition according to any one of claims 1 to 5, wherein the film forming polymer is selected from one or more of cellulose derivatives; polyhydric alcohols; saccharides, polysaccharides, gums and derivatives thereof; vinyl derivatives, polymers, copolymers or mixtures thereof; maleic acid copolymers; polyalkylene oxides or copolymers thereof; acrylic acid polymers and acrylic acid derivatives; fat; wax; fatty acid; fatty acid ester; long chain monohydric alcohol or their ester; or mixtures thereof.

8. The pharmaceutical composition according to any one of claims 1 to 5, wherein the plasticizer is selected from, polyethylene glycol (PEG), PEG 400, propylene glycol, glycerol, triethyl citrate and polysorbate.

9. The pharmaceutical composition according to claim 7, wherein the film forming polymer is selected from one or more of (a) the cellulosic derivatives are selected from one or more of ethyl cellulose, methylcellulose, hydroxypropylmethylcellulose (HPMC), Methocel E15, Methocel K15, hydroxypropyl cellulose (HPC), hydroxyethyl cellulose, hydroxymethyl cellulose, hydroxypropyl ethylcellulose, carboxymethylethyl cellulose, carboxyethylcellulose, carboxymethyl hydroxyethylcellulose, hydroxyethylmethyl carboxymethyl cellulose, hydroxyethyl methyl cellulose, carboxymethyl cellulose (CMC), methylhydroxyethyl cellulose, methylhydroxypropyl cellulose, carboxymethyl sulfoethyl cellulose, sodium carboxymethyl cellulose, and combinations thereof, (b) polysaccharides such as Sodium alginate and like, (c) gums such as Xanthan gum and like, and (d) polymers such as Eudragit EPO.

10. The pharmaceutical composition according to any one of claims 1 to 9, wherein the weight ratio of the plasticizer to the film forming polymer is in the range of about 1:3 to about 1:6.

11. The pharmaceutical composition according to any one of claims 1 to 5, wherein the pharmaceutically acceptable excipient or any other suitable and required auxiliary fil forming excipients includes an aqueous solvent, organic solvent, a base, a buffer, a sweetener, a colour additive, a flavouring agent, a preservative or mixtures thereof.

12. The pharmaceutical composition according to claim 11, wherein the pharmaceutically acceptable excipient is selected from one or more of (a) organic solvent, selected from the group of acetone, alcohol, ethanol, benzyl alcohol, benzyl benzoate, propylene glycol, butylene glycol, dibutyl phthalate, diethyl phthalate, dimethyl phthalate, dimethyl sulfoxide, dimethylacetamide, glycofurol, glycerin, isopropyl alcohol, isopropyl myristate, isopropyl palmitate, polyethylene glycol, propylene carbonate, pyrrolidone or mixtures thereof, (b) buffer is selected from the group of citric acid monohydrate, citrate, phosphate, succinate, tartrate, fumarate, gluconate, oxalate, lactate, acetate, histidine, sodium hydroxide or mixtures thereof, (c) the sweetener is selected from sucrose, mannitol, sucralose, aspartame and acesulfame, (d) the flavouring agent is selected from menthol, citric acid, lecithin, vanilla essence, peppermint essence and apple essence, (e) the preservative is selected from the group of sodium methyl hydroxyl benzoate, propyl hydroxyl benzoate, sorbic acid, paraben, bronopol, imidurea, phenoxyethanol, phenylmercuric acetate, benzyl alcohol, phenylmercuric borate, chlorocresol, benzethonium chloride, phenylethyl alcohol, benzalkonium chloride, hexetidine, chlorobutanol, cresol, cetylpyridinium chloride, phenylmercuric nitrate, chloroxylenol, propionic acid, phenol, thimerosal, sulfur dioxide, boric acid, edetic acid, sodium propionate, calcium chloride, sodium acetate, sodium sulfite, monothioglycerol, cetrimide, calcium acetate, butylene glycol, sodium metabisulfite, alcohol, propyl gallate, potassium metabisulfite, sodium lactate, chlorhexidine, calcium lactate, pentetic acid, propylene glycol alginate, sodium borate, magnesium trisilicate, isopropyl alcohol, dimethyl ether, butylated hydroxyanisole, pyrrolidone, lactic acid, dimethyl sulfoxide, or mixtures thereof, and (f) the colour additives are FD &C red # 40 and like.

13. A process for preparing the pharmaceutical composition of levothyroxine or its salt in the form of oral film, the process comprising the steps of:
a) add weighed quantity of Levothyroxine sodium to vehicle phase/solvents and mix for 15-30 min to get clear solution.
b) add weighed quantity of polymers to step (a) solution and mix for 30-45 min.
c) add weighed quantity of plasticizer and other excipients to step (b) solution and mix for 15-20 min.
d) observe Step (c) solution for clarity and cast on plain glass surface.
e) dry Step (d) casted solution in an oven at 30-40° C. to forms non-sticky films and pack properly.

14. A process for preparing the pharmaceutical composition of levothyroxine or its salt in the form of oral film, the process comprising the steps of:
a) adding weighed quantity of levothyroxine or its salt to solvent or mix of solvents and mixing for 15-30 min to get clear solution.
b) adding weighed quantity of polymers to step (a)′solution and mixing for 30-45 min. alternatively, polymer can be dissolved and dispersed separately in another portion of solvent mix.
c) adding weighed quantity of plasticizer and other pharmaceutically acceptable excipients to step (b) solution and mixing for 15-20 min.
d) spraying of the solution using roll or slot-die coating process followed by drying, lamination, and pouch packaging.
e) dry and/or wet bond lamination, and curing of the films can be done as required and deemed necessary.
f) drying of the prepared films can be done by roll support, Belt and/or air flotation methods.
g) alternatively, lab scale preparation can be prepared by casting of Step (c) solution on plain glass surface.
h) drying of Step(d) casted solution in an oven at 30° C.-70° C. to form non-sticky films and finally packing it into a pouch, to obtain a pharmaceutical composition of levothyroxine or its salt in the form of oral film.

15. The pharmaceutical composition according to any one of claims 1 to 5, wherein the oral film is used as and selected from various classes of films such as and including a mouth dissolving or soluble film, non-mucoadhesive film, fast disintegrating film, buccal film, mucoadhesive film, sublingual film, edible film or wafer.

16. The pharmaceutical composition according to any one of claims 1 to 5, wherein the oral film has surface area in the range of about 0.5 cm² to about 50 cm².

17. The pharmaceutical composition according to any one of claims 1 to 5, wherein the oral film has a thickness of about 0.5 mm to about 5 mm.

18. A pharmaceutical composition in the form of buccal film, wherein each buccal film comprises: (a) Levothyroxine Sodium, (b) Methocel E15, (c) Propylene glycol, (d) FD &C red # 40, (e) Peppermint flavour, (f) Ethanol, and (g) Purified water.

19. A pharmaceutical composition in the form of buccal film, wherein each buccal film comprises: (a) Levothyroxine Sodium, (b) Sodium alginate, (c) Glycerin, (d) FD &C red # 40, (e) Peppermint flavour, (f) Ethanol, and (g) Purified water.

20. A pharmaceutical composition in the form of sublingual film, wherein each sublingual film comprises: (a) Levothyroxine Sodium, (b) Sodium carboxymethyl cellulose, (c) Propylene glycol, (d) FD &C red # 40, (e) Peppermint flavour, (f) Ethanol, and (g) Purified water.

21. A pharmaceutical composition in the form of sublingual film, wherein each sublingual film comprises: (a) Levothyroxine Sodium, (b) Hydroxyethyl cellulose, (c) PEG 400, (d) FD &C red # 40, (e) Peppermint flavour, (f) Ethanol, and (g) Purified water.

22. A pharmaceutical composition in the form of mucoadhesive film, wherein each mucoadhesive film comprises: (a) Levothyroxine Sodium, (b) Methocel k15, (c) Propylene glycol, (d) FD &C red # 40, (e) Peppermint flavour, (f) Ethanol, and (g) Purified water.

23. A pharmaceutical composition in the form of mucoadhesive film, wherein each mucoadhesive film comprises: (a) Levothyroxine Sodium, (b) Sodium carboxymethyl cellulose, (c) Povidone K 30, (d) Glycerin (e) FD &C red # 40, (f) Peppermint flavour, and (g) Ethanol.

24. The composition according to any one of claims 17 to 22, comprising active drug potency (assay) preferably between 95%- 105% even after environmental exposure of one or more of the following:
(i) storage for one to three months at a temperature of 40° C. (±2° C.) and a relative humidity of 75% (± 5%); and
(ii) storage for three to twelve months at a temperature of 25° C. (±2° C.) and a relative humidity of 65% (± 5%).

25. The composition according to any one of claims 17 to 22, comprising less than about 2% total impurities following one or more of the following:
(i) storage for one to three months at a temperature of 40° C. (±2° C.) and a relative humidity of 75% (± 5%); and
(ii) storage for three to twelve months at a temperature of 25° C. (±2° C.) and a relative humidity of 65% (± 5%).

26. The composition according to any one of claims 17 to 22, comprising disintegration not more than 1 minute following one or more of the following:
(i) storage for one to three months at a temperature of 40° C. (±2° C.) and a relative humidity of 75% (± 5%); and
(ii) storage for three to twelve months at a temperature of 25° C. (±2° C.) and a relative humidity of 65% (± 5%).
